(19) United States
(12) Patent Application Publication
(10) Pub. No.: US 2022/0047214 A1
(43) Pub. Date: Feb. 17, 2022

(54) WEARABLE MONITOR DEVICE

(71) Applicant: Charles Edwards, Carencro, LA (US)

(72) Inventor: Charles Edwards, Carencro, LA (US)

(21) Appl. No.: 17/397,052

(22) Filed: Aug. 9, 2021

Related U.S. Application Data
(60) Provisional application No. 63/065,911, filed on Aug. 14, 2020.

(51) Int. Cl. A61B 5/00; A61B 5/022; A61B 5/0205; A61B 5/024; A61B 7/04; G06F 21/62

(52) U.S. Cl. CPC A61B 5/681; A61B 5/6898; A61B 5/02233; A61B 5/742; A61B 5/0205; G06F 21/6245; A61B 7/04; A61B 5/748; A61B 5/7465; A61B 5/0022; A61B 5/02438

(57) ABSTRACT

A wearable monitor device has a monitor having a mobile cell phone with a video camera configured to be worn on a wearer's wrist. The wearable monitor device further has a plurality of ports on the monitor for connecting health monitoring devices and a pair of clips on the monitor configured to attach the monitor to a pressure cuff or a wearable strap.

### FIELD OF THE INVENTION

The present invention relates to a wearable monitor that incorporates an ability to monitor one's blood pressure, heart rate, temperature among other things while providing a telephonic and video capability.

### BACKGROUND OF THE INVENTION

Historically, it is important to monitor one's health. There are many wearable monitoring systems available, such as smartwatches and fitness watches. These smartwatches have the capability of monitoring the wearer's heart rate allowing runners and joggers to keep track of their number of steps and other features for health reasons. Interestingly, however, the smartwatches while allowing audio have no video capability to date as the size of the watch is sufficiently small that it makes it difficult to incorporate such features.

The present invention solves these problems in a new and unique way and provides the wearer the ability to monitor his own vital signs and to transmit that information using an application software that will enable a doctor to see the measured results in real time.

### SUMMARY OF THE INVENTION

A wearable monitor device has a monitor having a mobile cell phone with a video camera configured to be worn on a wearer's wrist. The wearable monitor device further has a plurality of ports on the monitor for connecting health monitoring devices and a pair of clips on the monitor configured to attach the monitor to a pressure cuff or a wearable strap.

The wearable strap having a pair of openings, each opening to receive one of the pair of clips to affix the wearable monitor to the strap. The wearable monitor device further has a blood pressure cuff for wearing about a wearer's wrist. The blood pressure cuff is configured to attach to either the strap holding the monitor or directly to monitor. A heart rate device with a cord for attachment to a heart rate port of the plurality of ports of the monitor is provided. A stethoscope device with a cord for attachment to a stethoscope port of the plurality of ports of the monitor is provided.

The wearable straps have a pair of elongated straps with a hook and loop portion configured to wrap about a wearer's wrist. The pressure cuff has a hook and loop portion to attach to the wearable strap. Preferably, the wearable monitor has a touch screen. The wearable monitor also can have a touch pad with discrete keys. Preferably, the wearable monitor has a microprocessor and a programmable computer. The wearable monitor device includes a software application configured to be downloaded to the computer in the monitor. The wearable monitor has an encryption program to convert data and messaging to be sent wirelessly to a physician or assistant. The wearable monitor software is HIPAA compliant. The pressure cuff has a compliant flexible spring structure having a longitudinal opening or slot to fit onto a wrist portion of the wearer and be held in place by the flexible spring structure.

A method of providing and transmitting vital health data of a patient has the steps of: providing a monitor having a mobile cell phone with a video camera configured to be worn on a wearer's wrist with a plurality of ports on the monitor for connecting health monitoring devices; attaching a device to one of the ports and capturing a vital sign data; and sending the data to the monitor transmitting the data via the cell phone. The method further has the step of calling and initiating a video call by activating the camera on the monitor and calling a third party who can receive the transmitted data.

### BRIEF DESCRIPTION OF THE DRAWINGS

The invention will be described by way of example and with reference to the accompanying drawings in which:

FIG. 1 is a front view of wearable monitor device of the first embodiment of the present invention.

FIG. 2 is a side view of cantilever pull clamp clips that keeps device in place of the present invention.

FIG. 3 is a rear view of cantilever pull clamp clips that secure the device to outer cuff of the present invention.

FIG. 4 is an end view of the wearable monitor showing the pulse rate monitor, blood pressure, stethoscope and power ports of the device.

FIG. 5 is an end view of a first embodiment of the present invention.

FIG. 6 is a side view of Finger Monitor of present invention for attachment to the wearable monitor.

FIG. 7 is a top view of stethoscope with an optional EKG monitor of present invention for attachment to the wearable monitor.

FIG. 8 is a top view of a strap of the present invention for holding the wearable monitor.

FIG. 9 is a rear view of the back material of a pressure cuff of the present invention.

FIG. 10 is a top view of the wearable monitor device attached to the strap of the present invention along with the blood pressure cuff.

FIG. 11 is a perspective view of the second embodiment wearable monitor device being worn.

FIG. 12 is a perspective view of the second embodiment wearable monitor device attached to the blood pressure cuff of the present invention.

FIG. 13A is a perspective view of the second embodiment device.

FIG. 13B is a front view of the display and control panel of the second embodiment device with a wearable monitor device attached to a pressure cuff.

FIG. 14 is a top view of a full bar touch screen display of the third embodiment device.

FIG. 15 is an end perspective view of the overextended cuff and touch screen of the third embodiment device.

FIG. 16 is a top view of the second embodiment device with compact controls attached to the strap and blood pressure cuff.

FIG. 17 is a side view taken from FIG. 16.

### DETAILED DESCRIPTION OF THE INVENTION

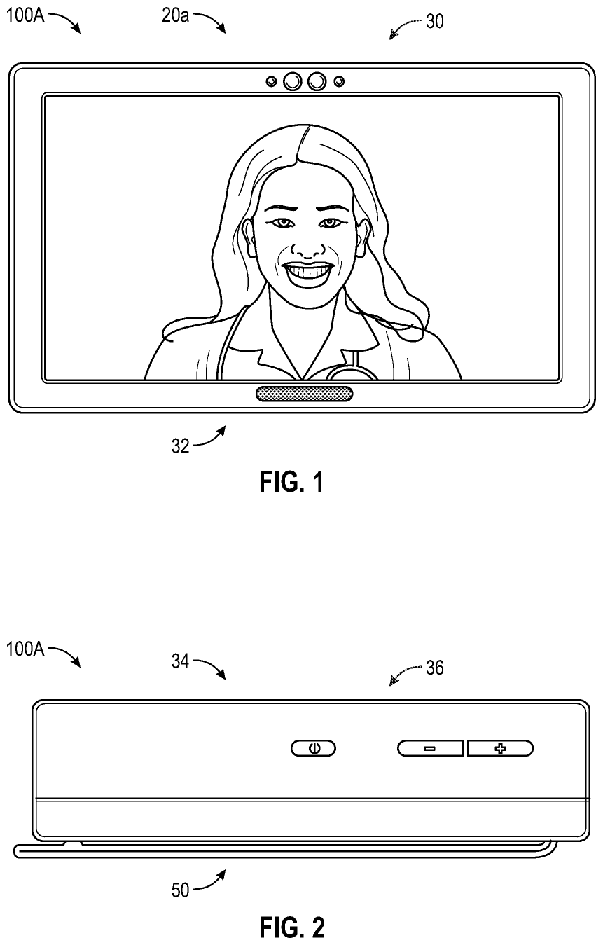

With reference to FIGS. 1-4, the wearable monitor device 100A of the present invention is shown. In FIG. 1, the monitor 100A with a touch screen video display 20A is illustrated. The touch screen video display 20A allows the wearer to make contact and video chat with other people. The advantage of this is that, should the wearer of the device need to talk to a physician, he or she could do so simply by dialing the phone number and initiating a video chat.

FIG. 2 is a side view of the wearable monitor device 100A showing a power button 34 and volume adjustment buttons 36 and cantilever pull clamp clips 50 that allow the device 100A to hold the device in place when fastened to a cloth or other material.

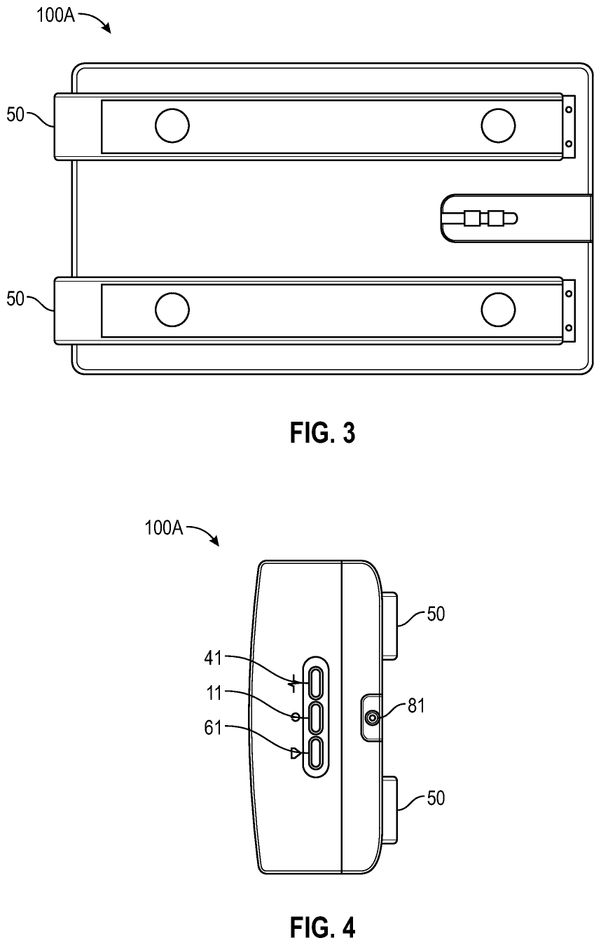

FIG. 3 shows a bottom view showing both cantilever pull clamp clips 50 that enable the monitor 100A to be attached to other devices if so needed.

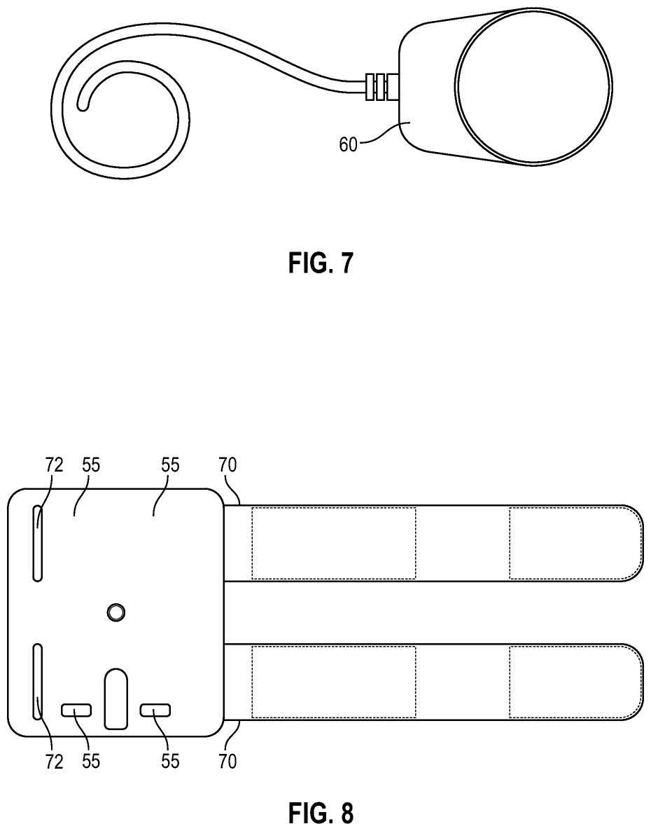

FIG. 4 is an end view of the device 100A showing a power port 11 for charging the wearable monitor 100A, a pulse rate monitor port 41, a blood pressure port 81 and a stethoscope port 61. These ports enable other devices to be attached. These other devices as shown in FIGS. 6 and 7 include a finger monitor 40 for measuring one's pulse or heart rate and a stethoscope 60 for measuring heart rate and breathing. The ports could also be used for an EKG type monitor separately or in combination with the stethoscope 60.

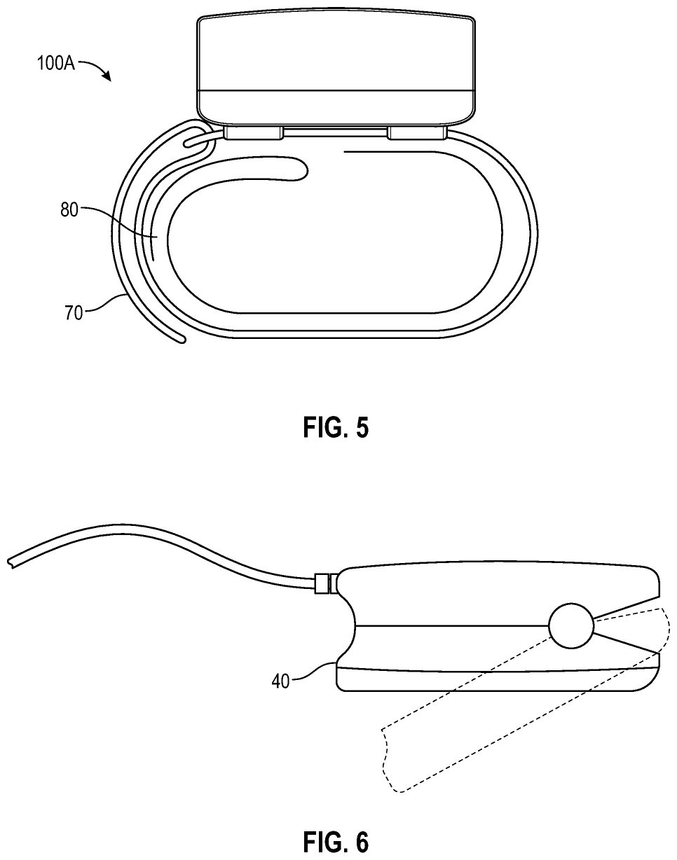

FIG. 5 shows an end view of the wearable monitor 100A attached to a pressure cuff device 80. The pressure cuff device 80 is a flexible member that, as illustrated, has two components. The first component being a strap 70 that enables the cantilever pull clamp clips 50 to slip through openings 55 illustrated in FIG. 8. The holes enable the wearable monitor to be affixed directly to the strap and provide a means for securing the device about one's arm. As shown, two elongated straps 70 are provided, these may use hook and loop fasteners and can be attached through openings 72 and allow the device to encircle one's arm when worn.

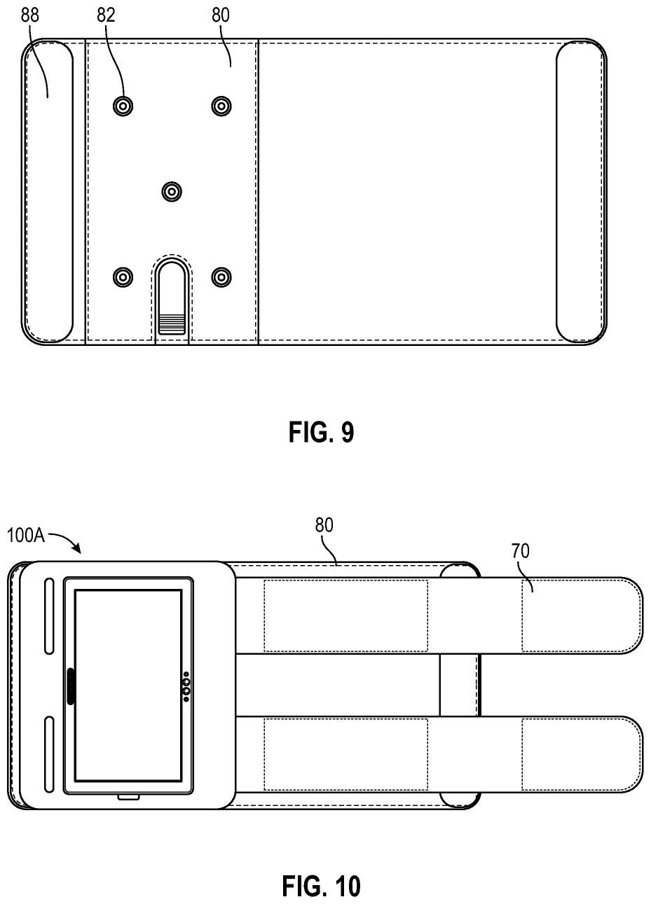

In FIG. 9 a pressure cuff 80 is shown in a laid out flat position. This pressure cuff 80 has reinforced material and rivet connections 82 to connect to the monitor device 100A. The material has the loop back material 88 for hook and loop attachment on one side as illustrated.

In FIG. 10, the wearable monitor 100A is shown affixed to the strap 70 and pressure cuff 80 as illustrated.

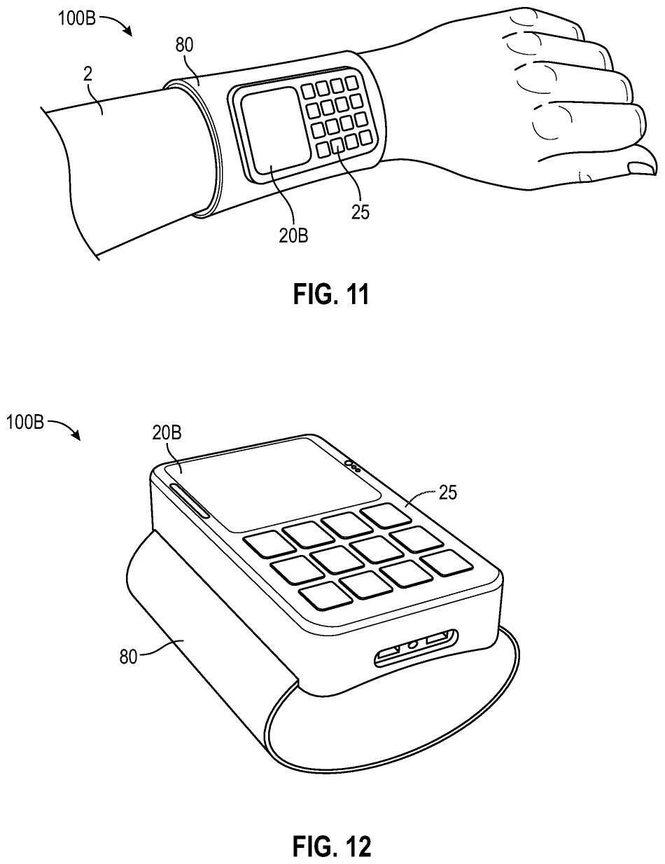

In FIG. 11, a second embodiment 100B of the wearable monitor device is shown looped about a wearer's arm 2. In this position, the pressure cuff can be activated to measure the wearer's blood pressure and heart rate. A signal from the pressure cuff is transmitted directly to the wearable monitor in such a fashion that one's blood pressure can be securely acquired through the device 100B.

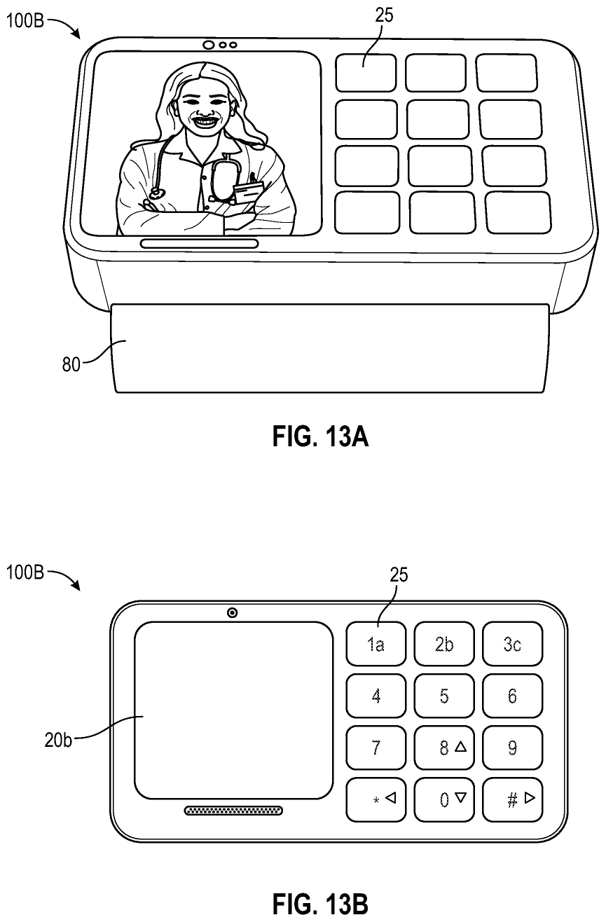

With reference to FIG. 12, the second embodiment monitor device 100B is illustrated in a perspective view. As shown, the wearable monitor 100B, in this case, has a partial touch screen 20B with a control or keypad 25 for dialing numbers or for inputting information to the device 100B. This embodiment is further illustrated in FIGS. 13A and 13B. In FIGS. 11, 12 and 13A, the monitor 100B is shown affixed to the pressure cuff 80.

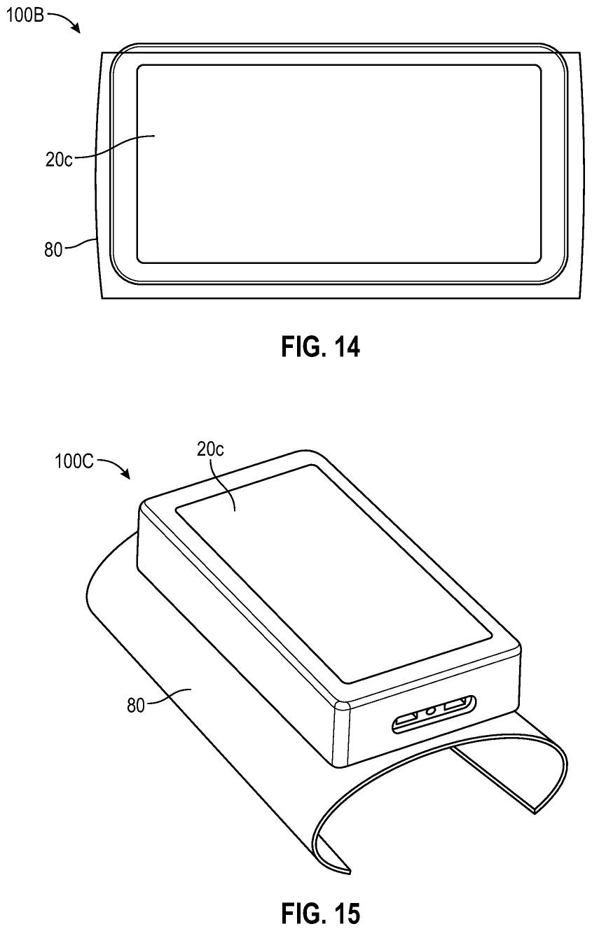

With reference to FIGS. 14 and 15, a third embodiment 100C of the device is shown wherein the pressure cuff 80 is shown made of a flexible material that is spring loaded and biased. This flexible material has either a soft leather, vegan leather or cloth fabric outer covering and is designed to snap over a wearer's arm. The wearable monitor 100C is affixed securely to the pressure cuff 80 in this third embodiment. As shown, the wearable monitor 100C has the full screen display 20C, alternatively it could use the second embodiment display 20B with the keypad 25 to attach to the pressure cuff 80.

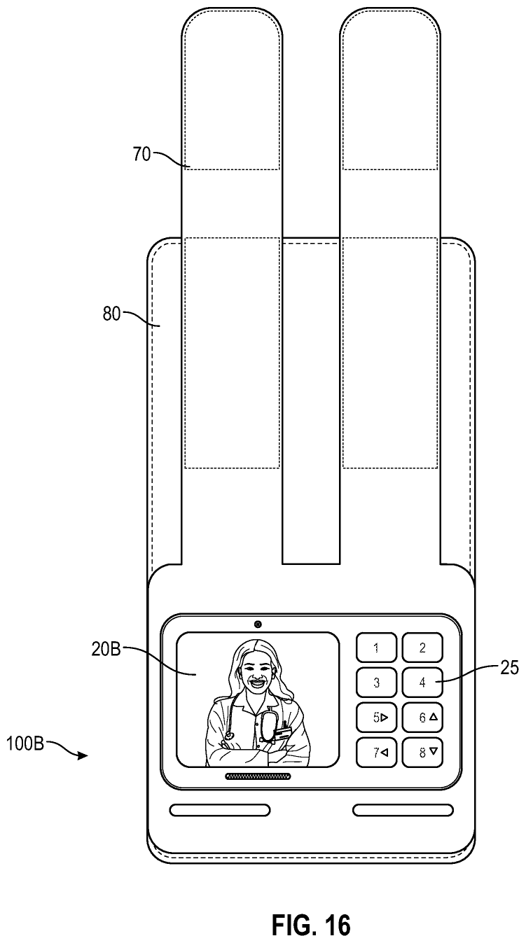

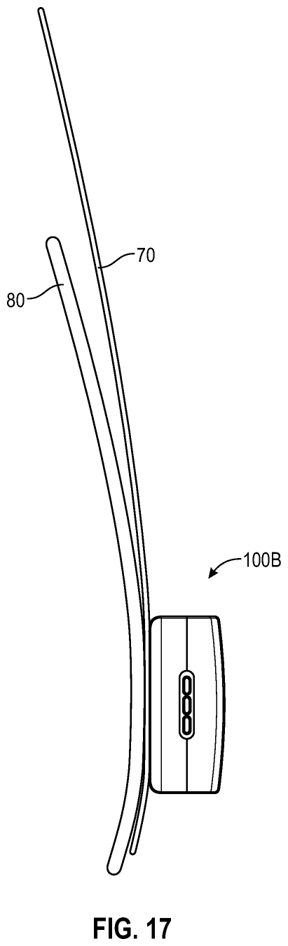

FIG. 16 shows the wearable monitor device 100B affixed to the straps 70 and pressure cuff 80. The straps have hook and loop closures to secure the pressure cuff about one's arm as previously discussed in the first embodiment.

In any of the embodiments, it is important to note that the vital signs of the patient can be monitored and securely held within the wearable monitor. The wearable monitor in all versions, has cell phone capability and Bluetooth connectivity and is capable of being downloaded with an application that allows the wearer to be able to download and transmit information to his or her physician and has a camera within the wearable monitor in each version that allows the physician to view the wearer/patient if so desired. This wearable monitor feature is unique to wrist mounted smart watches and other devices in that none currently are provided with the video capability although all have an audio capability. This is very important and can be quite useful in certain situations where the patient cannot visit the physician. It allows the physician to view and monitor the patient, check vital signs through the downloaded device and to get pressure readings in virtually real time from the patient. This is extremely helpful in situations where the patient may be experiencing a stroke or cardiac arrest and may need to be able to communicate rapidly prior to the situation deteriorating in such a fashion that the physician can make a determination of the best course of treatment for this particular patient. For example, in a heart attach situation, the patient may be instructed to take a low dose aspirin which they may or may not be carrying or it could be in the case of a diabetic where they may need to take an insulin dosage to counteract the onset of any reaction. These and other features are provided in the various embodiments of the present invention

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A wearable monitor device comprises:
a monitor having a mobile cell phone with a video camera configured to be worn on a wearer's wrist.

2. The wearable monitor device of claim 1 further comprises:
a plurality of ports for connecting health monitoring devices.

3. The wearable monitor device of claim 1 further comprises:
a pair of clips configured to attach the monitor to a pressure cuff or a wearable strap.

4. The wearable monitor device of claim 3 further comprises:
a wearable strap, the wearable strap having a pair of openings, each opening to receive one of the pair of clips to affix the wearable monitor to the strap.

5. The wearable monitor device of claim 1 further comprises:
a blood pressure cuff for wearing about a wearer's wrist.

6. The wearable monitor device of claim 4 further comprises:
a blood pressure cuff for wearing about a wearer's wrist.

7. The wearable monitor device of claim 6 wherein the blood pressure cuff is configured to attach to either the strap holding the monitor or directly to monitor.

8. The wearable monitor device of claim 2 further comprises:
a heart rate device with a cord for attachment to a heart rate port of the plurality of ports.

9. The wearable monitor device of claim 2 further comprises:
a stethoscope device with a cord for attachment to a stethoscope port of the plurality of ports.

10. The wearable monitor device of claim 4 wherein the wearable straps have a pair of elongated straps with a hook and loop portion configured to wrap about a wearer's wrist.

11. The wearable monitor device of claim 4 wherein the pressure cuff has a hook and loop portion to attach to the wearable strap.

12. The wearable monitor device of claim 1 wherein the wearable monitor has a touch screen.

13. The wearable monitor device of claim 12 wherein the wearable monitor has a touch pad with discrete keys.

14. The wearable monitor device of claim 1 wherein the wearable monitor has a micro processor and a programmable computer.

15. The wearable monitor device of claim 14 wherein the wearable monitor device includes a software application configured to be downloaded to the computer.

16. The wearable monitor device of claim 15 wherein the wearable monitor has an encryption program to convert data and messaging to be sent wirelessly to a physician or assistant.

17. The wearable monitor device of claim 16 wherein the wearable monitor software is HIPAA compliant.

18. The wearable monitor device of claim 3 wherein the pressure cuff has a compliant flexible spring structure having a longitudinal opening or slot to fit onto a wrist portion of the wearer and be held in place by the flexible spring structure.

19. A method of providing and transmitting vital health data of a patient comprises the steps of:
providing a monitor having a mobile cell phone with a video camera configured to be worn on a wearer's wrist with a plurality of ports on the monitor for connecting health monitoring devices;
attaching a device to one of the ports and capturing a vital sign data; and
sending the data to the monitor transmitting the data via the cell phone.

20. The method of claim 19 further comprises the step of:
calling and initiating a video call by activating the camera on the monitor and calling a third party who can receive the transmitted data.
